(19) United States
(12) Patent Application Publication
(10) Pub. No.: US 2026/0053985 A1
(43) Pub. Date: Feb. 26, 2026

(54) ENHANCED BIRTH TISSUE ALLOGRAFTS AND METHODS OF MANUFACTURE

(71) Applicant: ORBIO INNOVATIONS, LLC, Boulder, CO (US)

(72) Inventor: Mark D. Borden, Boulder, CO (US)

(21) Appl. No.: 19/306,874

(22) Filed: Aug. 21, 2025

Related U.S. Application Data
(60) Provisional application No. 63/685,515, filed on Aug. 21, 2024.

(51) Int. Cl. A61L 27/36

(52) U.S. Cl. CPC A61L 27/3633; A61L 27/3687; A61L 27/3691; A61L 2300/252

(57) ABSTRACT

Provided herein are a method of processing human birth tissue to create a variety of tissue forms that maintain high levels of beneficial compounds and increase tissue availability. Also provided are products made from the processed tissue forms, and application of these products in soft tissue repair and the treatment of wounds.

### CROSS REFERENCE TO RELATED APPLICATIONS

This application claims priority to U.S. Provisional Patent Application Ser. No. 63/685,515 filed Aug. 21, 2024, which is incorporated herein by reference in its entirety.

### FIELD

The present application relates to enhanced tissue repair compositions derived from human birth tissue and methods to manufacture such compositions.

### BACKGROUND

Allograft tissue products derived from human birth tissue (umbilical cord, amniotic sac, and placenta) are commonly used in a variety of soft tissue repair applications. This includes the treatment of various types of wounds such as full and partial thickness wounds, ulcers, burns, surgical wounds, and traumatic wounds. This also includes applications such as tendon, ligament, and nerve repair. These tissue grafts have been shown to be effective in stimulating and supporting a soft tissue healing response due to the variety of beneficial compounds contained within the birth tissue. This includes variety of collagen molecules (Type I, III, and IV), growth factor proteins (epidermal growth factors—EGF, vascular endothelial growth factor—VEGF, transforming growth factor beta—TGF-β, placental growth factor-PIGF, insulin-like growth factor—IGF, etc.) and extracellular matrix compounds (ECM) (glycosaminoglycans, proteoglycans, fibronectin, laminin, etc.). When placed at a wound site, the birth tissue products aid the healing process by releasing these beneficial compounds directly to the wound site and acting as a scaffold that supports new issue formation.

It is with these benefits in mind, among others, that various embodiments of the present disclosure were conceived and developed.

### SUMMARY

In some aspects, the current disclosure encompasses a method of processing a birth tissue, comprising obtaining or having obtained the birth tissue; subjecting the birth tissue to a two-step decellularization process, where the first decellularization step involves exposing the birth tissue to a first decellularization solution to remove blood-based cells and/or blood materials to obtain a clean tissue, and the second decellularization step involves exposing the clean tissue to a second decellularization solution comprising an acid or a base to obtain a second step decellularization solution; followed by a neutralizing step where the second step decellularization solution is neutralized to a pH of 4-8 to obtain a neutralization solid and a neutralization liquid.

In some aspects, this method may further include an initial size reduction step for the birth tissue prior to the two-step decellularization process, where the size reduction comprises cutting, milling, blending, sonicating, homogenizing, micronizing, pulverizing, grating, or macerating techniques, or any combination thereof, to obtain a particulate form of birth tissue. The temperature of the birth tissue may be reduced through refrigeration or freezing prior to size reduction.

Additionally, the method may involve cleaning the birth tissue or its particulate form with a cleaning agent, where the cleaning agent can be sterile water or a sterile salt solution.

The birth tissue may be harvested from umbilical cord, amniotic sac, or placenta, or any combination thereof. The birth tissue may comprise placental globe, umbilical cord, umbilical cord vessels, umbilical cord blood, chorionic membrane, amniotic membrane, Wharton's jelly, amniotic fluid, or extracellular material, or any combination thereof. In some aspects, the birth tissue is derived from a human.

In some aspects, the first decellularization solution may comprise water, salt solutions, mild ionic detergents, enzymes, or alcohol solutions, or any combination thereof. The detergent may be Triton X-100. The enzymes may comprise trypsin, DNAase, collagenase, dispase, or lipase, or any combination thereof.

In some aspects, the second decellularization solution may comprise an acid solution comprising hydrochloric acid, acetic acid, citric acid, lactic acid, formic acid, or trifluoroacetic acid (TFA). In some aspects, the second decellularization solution may comprise a base solution comprising sodium hydroxide, ammonium hydroxide, potassium hydroxide, or calcium hydroxide. The acid or base concentration in the acid solution or the base solution may range from 0.01 M to 10 M.

In some aspects, the neutralization step may comprise a neutralizing acid or neutralizing base solution that comprises a base solution comprising sodium hydroxide, potassium hydroxide, calcium hydroxide, sodium carbonate, ammonium hydroxide, or any combination thereof, or an acid solution comprising hydrochloric acid, acetic acid, citric acid, lactic acid, formic acid, trifluoroacetic acid (TFA), or any combination thereof. The neutralizing acid or neutralizing base solution may have an acid concentration or base concentration respectively ranging from 0.01 M to 10 M. The neutralization step may comprise, or further comprise, a neutralization process utilizing dialysis, diafiltration, or other means.

The neutralization solid may comprise a collagen precipitate and/or a birth tissue particulate. The neutralization liquid may comprise beneficial compounds such as soluble gelatin, growth factors, proteins, or extracellular matrix (ECM) compounds, individually or in combination. The method may further comprise drying the soluble gelatin, growth factors, proteins, or ECM, or any combination thereof, and optionally incorporating the dried gelatin, growth factors, proteins, or ECM, or any combination thereof into an allograft tissue composition.

The method may include subjecting the neutralization liquid to protein purification or desalting procedures, or combinations thereof. The method may comprise separating the neutralization solid from the neutralization liquid. The neutralization solid and/or the neutralization liquid may be incorporated into an allograft tissue composition. In some aspects, the neutralization solid and/or the neutralization liquid may be dried, and optionally incorporated into an allograft tissue composition. The drying methods may comprise freeze-drying, air drying, or dehydration.

In some aspects, the allograft tissue composition may be in sheet, powder, gel, paste form, or any combination thereof.

The allograft tissue composition may have increased solubility for one or more of collagen, gelatin, growth factors, or extracellular matrix compounds. The composition may release the growth factors and/or extracellular matrix compounds within less than about 1 hour to less than about 8 hours. The composition may release the growth factors and/or extracellular matrix compounds with a biphasic release rate comprising a first fast phase and a subsequent slow phase for release. In some aspects, the fast phase may last less than about 1 hour to less than about 8 hours, and the slow phase may last greater than about 2 days to greater than 6 months.

The allograft tissue composition may be processed to a dried sheet. The dried sheet may be further processed into a powder through blending, grinding, or milling techniques, or combinations thereof. The allograft tissue composition may further comprise an amniotic membrane. The allograft tissue composition may be added to a collagen or gelatin wound product.

In some aspects, the current disclosure encompasses an allograft tissue composition comprising a processed birth tissue, wherein the birth tissue is processed using a method including obtaining or having obtained the birth tissue, subjecting it to a two-step decellularization process involving a first step to remove blood-based cells and/or blood materials to obtain a clean tissue, and a second step where the clean tissue is exposed to a solution comprising an acid or a base, followed by neutralizing to a pH pf 4-8 to obtain a neutralization solid and a neutralization liquid. The processed birth tissue is obtained from the neutralization solid, the neutralization liquid, or both.

In some aspects, the neutralization solid may comprise a collagen precipitate and/or residual birth tissue particulate. In some aspects, the neutralization liquid may comprise one or more beneficial compounds including soluble gelatin, growth factors, proteins, or extracellular matrix compounds, or any combination thereof.

The method can include separating the neutralization solid from the neutralization liquid. One or both of the neutralization solid and neutralization liquid may be incorporated into the allograft tissue composition. The neutralization solid and/or liquid may be dried, with incorporation of the dried components into the allograft tissue composition. The drying may involve freeze-drying, air drying, or dehydration.

The allograft tissue composition may be in the form of a sheet, powder, gel, paste, or any combination thereof. The allograft tissue composition may have increased solubility for one or more of collagen, gelatin, growth factors, and extracellular matrix compounds. The allograft tissue composition may release the growth factors and/or extracellular matrix compounds within less than about 1 hour to less than about 8 hours. The allograft tissue composition may release the growth factors and/or extracellular matrix compounds with a biphasic release rate comprising a first fast phase and subsequent slow phase. The fast phase may last less than about 1 hour to less than about 8 hours. The slow phase may last greater than about 2 days to greater than 6 months.

Additionally, allograft tissue composition can be processed to a dried sheet. The dried sheet may be further processed into a powder, through blending, grinding, or milling techniques, or any combination thereof.

The allograft tissue composition may further comprise an amniotic membrane. The allograft tissue composition may be added to a collagen or gelatin wound product.

In some aspects, the current disclosure encompasses a birth tissue allograft product, comprising one or more of extracellular matrix, collagen, growth factors, or gelatin, where these components are obtained from human birth tissue using a two-step decellularization process. The two-step process may involve exposing the tissue first to a decellularization solution to remove blood-based cells and/or blood materials to obtain a clean tissue, and then to a second decellularization solution comprising an acid or a base to obtain a second step decellularization solution. The decellularization process may further include neutralizing the second step decellularization solution to a pH of 4-8 using a neutralizing step. In some aspects, the neutralization step may comprise use of a neutralizing acid or a neutralizing base solution and/or a process comprising dialysis, diafiltration, or other means.

The birth tissue may include placental globe, umbilical cord, umbilical cord vessels, umbilical cord blood, chorionic membrane, amniotic membrane, Wharton's jelly, amniotic fluid, or extracellular material.

The birth tissue allograft product may have increased solubility for growth factors and/or extracellular matrix compounds. The birth tissue allograft product may release the growth factors and/or extracellular matrix compounds within less than about 1 hour to less than about 8 hours. The birth tissue allograft product may have a biphasic release rate, with a first fast phase and subsequent slow phase for growth factors and/or extracellular matrix compounds. The fast phase may last less than about 1 hour to less than about 8 hours. The slow phase may last for greater than about 2 days to greater than 6 months.

The birth tissue allograft product may be in sheet, powder, gel, paste form, or any combination thereof. The product may further include an amniotic membrane. The product may be added to a collagen or gelatin wound product. The product may comprise various allograft tissue compositions such as a placental collagen-growth factor-extracellular matrix sheet, a placental collagen-growth factor-ECM powder, an amniotic membrane coated with placental collagen, growth factors, and/or ECM, a placental gelatin sheet, a placental gelatin sheet powder, a collagen or gelatin wound product enhanced with placental collagen, growth factors, and ECM, or a growth factor-ECM foam or particulate.

### BRIEF DESCRIPTION OF THE DRAWINGS

The following drawings form part of the present specification and are included to further demonstrate certain aspects of the present disclosure. Aspects of the present disclosure may be better understood by reference to one or more of these drawings in combination with the detailed description of specific aspects presented herein.

FIG. 1 shows a flowchart of a birth tissue process conducted on the placenta and amniotic sac.

FIG. 2 shows photographs of a placental tissue process utilizing a two-step decellularization process and the resulting neutralization materials formed from the process.

FIG. 3 provides photographs of the freeze-dried materials created from the liquid and solid neutralization materials.

FIG. 4 shows birth tissue allograft products that can be created from various combinations of amniotic membrane and placenta connective tissue matrix materials.

FIG. 5 shows birth tissue allograft products that can be created from various placental connective tissue matrix materials.

FIG. 6 shows photographs of a birth tissue allograft product composed of amniotic membrane and placental connective tissue matrix materials and its associated hydrated handling.

FIG. 7 shows photographs of a placental collagen-growth factor-ECM powder and its associated hydrated handling.

### DETAILED DESCRIPTION

It will be appreciated that numerous specific details are set forth in order to provide a thorough understanding of the examples described herein. However, it will be understood by those of ordinary skill in the art that the examples described herein can be practiced without these specific details. In other instances, methods, procedures, and components have not been described in detail so as not to obscure the related relevant feature being described. Also, the description is not to be considered as limiting the scope of the examples described herein. The drawings are not necessarily to scale, and the proportions of certain parts may be exaggerated to better illustrate details and features of the present disclosure.

Disclosed herein are processing methods to create a variety of birth tissue allograft compositions comprised of birth-tissue derived collagen, gelatin, growth factors, and/or ECM materials. In some embodiments, with the use of a 2-step decellularization process, various allograft forms are envisioned to improve soft tissue healing and enhance the biological response by maintaining high levels of growth factors and ECM, and increasing availability of said growth factors.

In some aspects, the current disclosure is based on the observation that currently used methods to prepare allograft products from birth tissue lack sufficient availability (i.e., solubility) and/or flexibility of release of beneficial biomaterials. The primary type of birth tissue graft on the market consists of amniotic membrane harvested from the umbilical cord, amniotic sac, and/or placenta, while the remaining material, for example, the placenta is discarded. Amniotic tissue is a barrier membrane consisting of an inner layer (amnion) and an outer layer (chorion). Once isolated from the birth tissue, the amniotic membrane is then subject to additional processing and eventually dried in a single layer form, or stacked on itself in a multi-layer form. Dried membranes are then cut to sizes appropriate to their clinical application and sterilized.

By discarding the placenta, a large source of additional collagen, growth factors, and ECM is lost. While the amnion contains these compounds, the amount is limited due to the total weight of the amniotic membrane. Additionally, the biological activity of the compounds and the amniotic membrane can be reduced, due to aggressive processing methods. Additionally, although amniotic membrane allograft is the primary birth tissue allograft form used clinically, the amniotic membrane is a relatively thin tissue (20-50 μm) with an average total wet tissue weight of approximately 1-3 g. Due to this small size and mass, the amount of beneficial materials is also small. Conversely, the placenta is a much larger tissue with an average total wet weight of 200-500 g, following amniotic membrane removal. Similar to the amniotic membrane, the placenta also contains beneficial collagen, growth factors, and ECM.

Due to the larger size, the placenta can be processed to provide a greater amount of birth tissue allograft products with higher growth factor content, compared to amniotic membrane products. As such, some current products are derived from placental tissue. These types of allograft tissues are called placental connective tissue matrix (pCTM) and are typically provided in a powder or sheet form. Placental products are intended to be used to supplement or replace damaged or inadequate soft tissue, and to fill soft tissue defects or voids created by injury or surgery. However, the methods used to create current pCTM products share some of the drawbacks of the amniotic membrane allografts products. In particular, pCTM processing can use harsh extraction conditions that can lead to diminished growth factor and ECM content. Further, current methods utilize a single decellularization step that keeps the remaining growth factors and ECM trapped in a cross-linked collagen matrix, which can delay their solubility and associated biological activity.

In soft tissue healing applications, the biological activity of the birth tissue is due to the presence of beneficial, water-soluble compounds (growth factors and ECM) within a cross-linked collagen matrix. While the collagen matrix provides a scaffold to support new tissue growth, the growth factors and other ECM will locally interact with cells to stimulate and support a cellular healing response. However, the resulting biological activity of the tissue is dependent on whether these compounds are immediately available to the cells or have to be slowly released over time from the cross-linked collagen matrix (i.e., delayed solubility). This concept is called bioavailability and is defined as the proportion of biologically active molecules that can immediately interact with local cells compared to compounds that are slowly released over time. In birth tissue, growth factors and ECM compounds on the surface of the tissue are readily soluble and can provide immediate interaction with cells. However, the majority molecules are embedded within the collagen matrix and are slowly released over time (delayed solubility). As a result, this gives standard birth tissue allograft a moderate degree of bioavailability.

In addition to release of beneficial molecules and associated bioavailability, the biological activity of birth tissue is also dependent on the amount of growth factors and ECM present in the fully processed tissue. Typical birth tissue processing involves a variety of steps which can directly impact whether the growth factor and ECM levels are maintained or reduced. Birth tissue allograft is subjected to various cleaning, disinfection, and decellularization steps. While these steps are needed to process the tissue into a non-immunogenic form, aggressive processing can negatively impact the biological activity of the tissue by reducing growth factor and ECM levels. In particular, solution processing can solubilize and extract beneficial molecules out of the tissue. Since these solutions are discarded during processing, the biological activity from the compounds is lost. Additionally, certain drying procedures (typically involving high heat or prolonged heating duration) can degrade and denature proteins. The net effect is that the biological activity of the birth tissue allograft can be reduced with processing.

In addition to birth tissue allografts, collagen-based products are also commonly used to treat soft tissue wounds and injuries. These products are primarily composed of Type I collagen or Type I/III collagen derived from bovine or porcine tendon and hides, respectively. Additionally, this collagen can be further processed into gelatin (fragments of the triple helix collagen molecular) or hydrolyzed collagen (fragments of gelatin) and used for the same soft tissue repair applications. Collagen products benefit the healing process by providing a scaffold for soft tissue formation and epithelialization, reducing inflammation, absorbing wound fluid, and aiding with clot formation (hemostasis). Gelatin aids in wound healing by absorbing liquid, maintaining hydrated wound bed, and providing hemostatic properties. However, due to the animal origin of these products, these collagen and gelatin materials are highly purified and do not contain growth factors or other ECM compounds which can actively stimulate and support the healing process.

Disclosed herein is a method that addresses these limitations by utilizing a two-step decellularization process that maintains high levels of beneficial compounds in the tissue and improves the availability of these compounds. In some aspects, these compounds may be used alone or combined with an amniotic membrane to produce an enhanced allograft products. The disclosed products and methods of making the same provide several benefits over the currently used products. First, they greatly surpass the amount of beneficial compounds available in the products due to the gentleness of the processing methods used to assemble them. Secondly, these products provide greater solubility and release rates of beneficial compounds (i.e., bioavailability), depending on the application. For example, for wound dressings, which require multiple dressing changes, it may be beneficial to release all the beneficial compounds within a short period of time. However, an implant may work better if these beneficial compounds have delayed release, or are released in a biphasic manner, with a quick phase, and a delayed release phase. The methods disclosed herein can be used to fine tune the product based on the application as is further elaborated below.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present disclosure or the appended claims.

Any term of degree such as, but not limited to, “substantially” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

The terms “comprising,” “including,” and “having” are used interchangeably in this disclosure. The terms “comprising,” “including,” and “having” mean to include, but not necessarily be limited to the things so described.

The terms “or” and “and/or,” as used herein, are to be interpreted as inclusive or meaning any one or any combination. Therefore, “A, B, or C” or “A, B, and/or C” mean any of the following: “A,” “B,” or “C”; “A and B”; “A and C”; “B and C”; “A, B, and C.” An exception to this definition will occur only when a combination of elements, functions, steps, or acts are in some way inherently mutually exclusive.

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (3rd ed. 2006); Chambers Dictionary of Science and Technology (Walker ed., 1999); The Glossary of Genetics, 5th Ed., R. Rieger et al. (2008), The Harper Collins Dictionary of Biology (1991), all of which are incorporated by reference herein. As used herein, the following terms have the meanings ascribed to them below, unless specified otherwise.

The phraseology and terminology employed herein are for the purpose of description and should not be regarded as limiting. When introducing elements of the present disclosure or the preferred aspects(s) thereof, the articles “a,” “an,” “the,” and “said” are intended to mean that there are one or more of the elements. The terms “comprising,” “including,” and “having” are intended to be inclusive and mean that there may be additional elements other than the listed elements. Wherever the terms “comprising” or “including” are used, it should be understood the disclosure also expressly contemplates and encompasses additional aspects “consisting of” the disclosed elements, in which additional elements other than the listed elements are not included.

The term “about” or “approximately,” as used herein, can mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” can mean an acceptable error range for the particular value, such as 10% of the value modified by the term “about.” As used herein, the term “about,” can mean relative to the recited value, e.g., amount, dose, temperature, time, percentage, etc., ±10%, ±9%, ±8%, ±7%, ±6%, ±5%, ±4%, ±3%, ±2%, or ±1%.

As used herein, the term “human birth tissue” includes, but is not limited to, elements of the placental organ such as, for example, placental globe, umbilical cord, umbilical cord vessels, umbilical cord blood, chorionic membrane, amniotic membrane, Wharton's jelly, amniotic fluid and other placental gelatins, fluids, cells, and extracellular material. In an embodiment, the birth tissue may be obtained from a healthy female.

As used herein “enhanced solubility” or “increased availability” or “enhanced availability” refers to the improved delivery and sustained presence of biologically active agents (e.g., growth factors, stem cells, peptides, or drugs) at the site of tissue repair, allowing for more effective biological action. The enhancement, as described herein may be with respect to other available birth tissue-based allograft tissue compositions. This enhancement can be achieved, for example through gentler processing methods and/or the increased solubility of growth factors and ECM molecule, as further described herein. In some aspects, the disclosed allograft tissue composition may have at least, at most, or about, 1.0, 2.0, 3.0, 4.0, 5.0, 6.0, 7.0, 8.0, 9.0, 10.0 (or any range derivable therein), fold higher availability compared to other commercial allograft tissue compositions. For example, in some aspects, the growth factors from the processed birth tissue allografts may be more available and/or transfuse out more readily from the ECM or collagen matrix, thus providing at least, at most, or about, 1.0, 2.0, 3.0, 4.0, 5.0, 6.0, 7.0, 8.0, 9.0, 10.0 (or any range derivable therein), fold higher availability of the growth factor compared to other commercial allograft tissue compositions.

As used herein “powder” refers to a birth tissue material form consisting of fragmented particles (also called particulate). The terms “powder”, “particles”, and “particulate” are used interchangeably. In some aspects, the particles may have micron or mm sizes, such as 25 μm, 50 μm, 100 μm, 250 μm, 500 μm, 750 μm, 1 mm, 2 mm, 3 mm, 4 mm, or larger than 4 mm.

### II. Method of Making

Human birth tissue may be recovered from a full-term vaginal or aseptic Cesarean delivery of a newborn. The placental globe, umbilical cord, umbilical cord vessels, umbilical cord blood, chorionic membrane, amniotic membrane, Wharton's jelly, amniotic fluid and other placental gelatins, fluids, cells, and/or extracellular material can be recovered from a female after the newborn is removed. In some embodiments, where only the amniotic membrane and the placental globe are selected for further processing, portions of the human birth tissue material (e.g., chorionic membrane or umbilical cord) may be removed and discarded by blunt dissection or excision. For example, the chorionic membrane may be removed by applying finger pressure and sliding it off of the amniotic membrane using as little pressure as possible to avoid tearing of the amnion.

All harvested donor tissues may be thoroughly screened through multiple processes to ensure safety. Initial donor selection may be done using any number of criteria known in the art. For example, health, age, multiple regulatory and ethical guidelines may be used to inform of donor selection. Infectious disease testing of donor blood specimens may be performed for each tissue donor on a specimen collected at the time of donation or within seven days prior to or after donation. Exemplary infectious disease testing includes, but is not limited to, antibodies to the human immunodeficiency virus, type 1 and type 2 (anti-HIV-1 and anti-HIV-2); nucleic acid test (NAT) for HIV-1; hepatitis B surface antigen (HBsAg); total antibodies to hepatitis B core antigen (anti-HBc-total, meaning IgG and IgM); antibodies to the hepatitis C virus (anti-HCV); NAT for HCV; antibodies to human T-lymphotropic virus type I and type II (anti-HTLV-I and anti-HTLV-II); and/or syphilis (a non-treponemal or treponemal-specific assay may be performed).

Following birth tissue harvest, the tissue is transferred to a tissue processing facility (tissue bank or tissue processor). During birth tissue processing, the birth tissue material may be initially rinsed to clean or remove any surface blood, debris, or cellular components. In an embodiment, the birth tissue material may be rinsed with sterile water. In an embodiment the materials may be rinsed with sterile saline solution. The sterile saline solution may comprise from about 0.9% to about 20% NaCl. In some embodiments, the sterile saline solution may comprise about 0.9%, 1%, 2%, 3%, 4%, 5%, 6%, 7%, 8%, 9%, 10%, 11%, 12%, 13%, 14%, 15%, 16%, 17%, 18%, 19%, or 20% (or any range derivable therein) of NaCl. According to an embodiment, the sterile saline solution comprises about 18% NaCl. According to one embodiment, this rinse step may be repeated at least once, at least twice, at least thrice, or more times.

In an embodiment, the birth tissue material or a portion thereof may be initially stored at a temperature between about 1° C. to about 10° C. for a period of less than, more than or about 1, 5, 10, 15, 20, 25, 30, 35, 40, 45, 50, 55, 60, 65, 70, 75, 80, 85, 90, 95, 100, 105, 110, 115, 120 hours (or any range derivable therein) prior to further processing. In an embodiment, the birth tissue material or a portion thereof may be frozen for an extended period of time (ranging from days, to weeks, or to months), subsequent to being rinsed. In an embodiment, the birth tissue material or a portion thereof may be frozen in liquid nitrogen or dry ice. In an embodiment, the birth tissue material or a portion thereof may be processed further prior to freezing or after freezing, for example by cutting, grating, milling, blending, sonicating, homogenizing, micronizing, pulverizing, macerating, or any combination thereof.

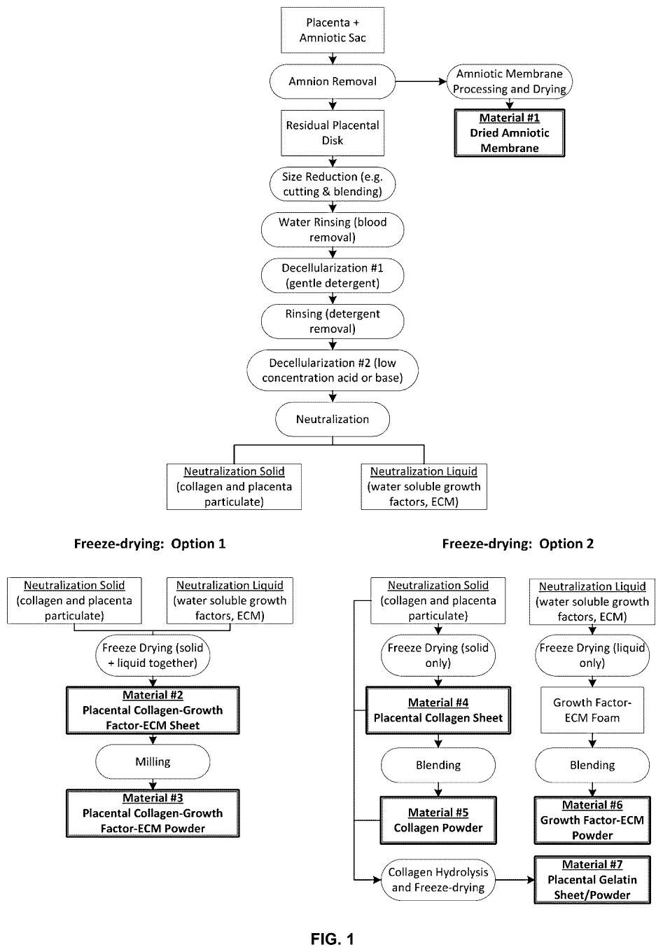

In an embodiment, the birth tissue material or a portion thereof may be the placenta. FIG. 1 provides an exemplary workflow for processing placental tissue per the current disclosure. The placenta may be initially processed to remove the amnion, and the remaining placental tissue may be processed or stored for later processing after an initial cleaning step as indicated above. The isolated amnion may be further processed to obtain dried amniotic membrane using standard amnion processing steps. Processing of the placental tissue may involve an initial size reduction step or tissue disruption step. This can be achieved by any method known in the art, non-limiting examples of which include cutting, grating, milling, blending, sonicating, homogenizing, micronizing, pulverizing, macerating, or any combination thereof. In an embodiment, the size reduction process can occur in sequence whereby the tissue is reduced to sequentially smaller sizes (e.g., cutting following by blending followed by homogenization). In an embodiment, the initial size reduction may be done by cutting the placenta into about 0.1 to about 1 inch squares, for example about 0.1 inch, about 0.2 inch, about 0.3 inch, about 0.4 inch, about 0.5 inch, about 0.6 inch, about 0.7 inch, about 0.8 inch, about 0.9 inch, or about 1 inch squares (or any range derivable therein), or any combination thereof. Alternatively, the placental tissue may be blended, milled or homogenized. In an embodiment, the placental tissue may be reduced in size by cutting, grating, milling, blending, sonicating, homogenizing, micronizing, pulverizing, macerating, or a sequential combination thereof. The particles of the resulting placental tissue composition may be centimeter, millimeter, or micron in size. In an embodiment, the size reduction is conducted on frozen or partially frozen tissue to aid in the size reduction process. In an embodiment, any one or more of these procedures can be conducted at room temperature, cold temperature (1° C. to about 10° C.) or under cryogenic conditions (<0° C.).

The resulting particulate tissue may be further cleaned. The particulate tissue may be further subjected to a rinsing step with a cleaning agent to remove surface blood, debris, and cellular components. In some embodiments, the cleaning agent is water. In some embodiments, the cleaning agent is 0.9%-20% saline solution. In some embodiments, the sterile saline solution may comprise about 0.9%, 1%, 2%, 3%, 4%, 5%, 6%, 7%, 8%, 9%, 10%, 11%, 12%, 13%, 14%, 15%, 16%, 17%, 18%, 19%, or 20% (or any range derivable therein) of NaCl. This is conducted until the rinse becomes clear with no discoloration due to the presence of blood.

In an embodiment, the method also encompasses a decellularization process comprising two or more decellularization steps. The process may be conducted on a complete set of birth tissue (umbilical cord, amniotic sac, and placenta) or one or more of the tissues. While a variety of processing techniques may be utilized, the birth tissue process broadly involves reducing the birth tissue (for example the placenta) into a particulate form (through cutting, grating, milling, blending, sonicating, homogenizing, micronizing, pulverizing, macerating, or any combination thereof, etc.) as provided above, rinsing the particulate tissue with water or saline to remove surface blood, debris, and cellular components, subjecting the rinsed tissue to an initial decellularization step using a gentle decellularization solution and discarding the solution and associated rinses, subjecting the particulate tissue to a second decellularization process using acids or bases, neutralizing the second decellularization solution with a neutralizing step, wherein the neutralization step may comprise one or more of, a neutralizing acid, or a neutralizing base solution, or a neutralizing process. In some aspects, the neutralizing process may comprise one or more of, dialysis, ion exchange, diafiltration, electrochemical methods, or any combination thereof. In some aspects, the neutralizing process is dialysis, or diafiltration. In some aspects, the method further comprises capturing the resultant neutralization solid and neutralization liquid, and drying the neutralization solid and neutralization liquid, either together or separately. A third decellularization process using acid or base solutions can also be designed to partially convert the neutralization solid into gelatin (fragmented collagen) or hydrolyzed collagen (gelatin fragments). Processed compounds can then be dried or freeze-dried to aid in the creation of beneficial birth tissue allograft products.

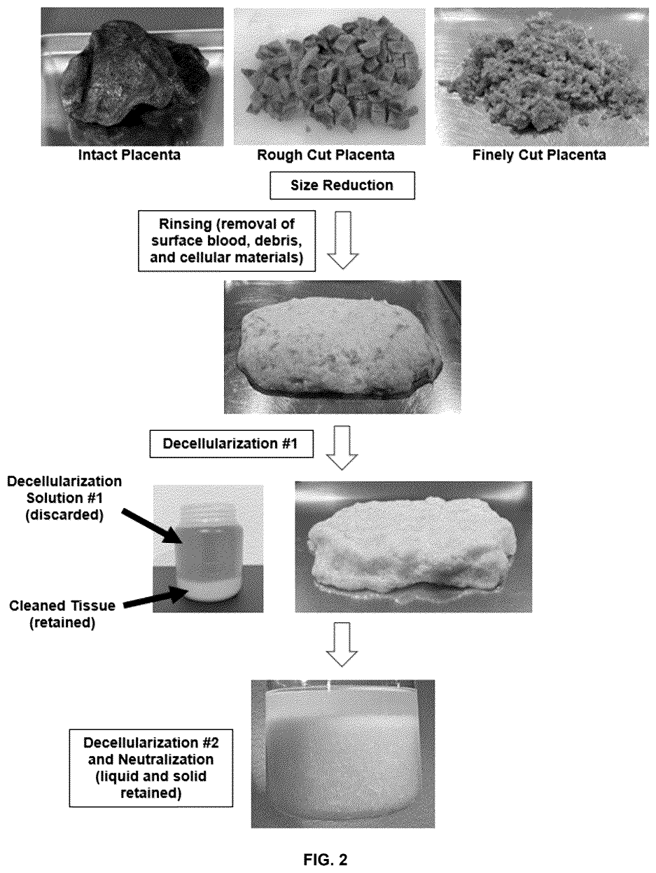

An example of a two-step decellularization process conducted on placental tissue is shown in FIG. 2. In this embodiment, the placental tissue is first processed to reduce the tissue into a particulate form. This is done to facilitate tissue cleaning and decellularization, and can be accomplished by various cutting, grating, milling, blending and/or homogenization procedures. To aid in the tissue size reduction, the starting tissue may be refrigerated, partially frozen, or fully frozen. The cold temperature can also aid in minimizing any protein denaturation or degradation resulting from the mechanical size reduction processes. Once the desired particulate size is reached, the tissue is subjected to a rinsing process initially using water, followed by an initial decellularization step using a gentle agent (for example, a mild detergent like Triton X-100). In some aspects, the concentration of Triton-X 100 may be anywhere between 0.5% to 20%, for example, 0.5%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, 15.5%, 16.0%, 16.5%, 17.0%, 17.5%, 18.0%, 18.5%, 19.0%, 19.5%, 20.0% (or any range derivable therein), depending on the tissue type and conditions used for cleaning. In some aspects, the Triton X-100 concentration for is 4%. During the initial decellularization, the tissue is suspended in the solution and mixed. Mixing can occur using mechanical or magnetic stirring, homogenization, agitation, or other means. The initial decellularization solution can be removed, fresh solution can be added to the tissue, and mixing can continue. Additionally, the tissue can be soaked with no mixing for an extended time, such as 16-24 hours. Throughout the initial decellularization, the solutions are removed using decanting techniques, centrifugation, sieving, or other liquid-solid separation methods. Since a gentle decellularization solution is chosen to minimize degradation to the tissue and reduce solubilization of beneficial materials, these solutions can be discarded. The initial decellularization continues and the process is repeated until the tissue takes on a tan/white appearance with an evident lack of red color due to the removal of residual blood. At this point, a cleaned particulate tissue is created. As needed, additional size reduction processing may be utilized.

Following the initial decellularization, the clean tissue particulate is subjected to a second decellularization step utilizing acid or base solutions. In an embodiment, any suitable acid or base may be used for this process. Non-limiting examples of suitable acids include hydrochloric acid, acetic acid, citric acid, lactic acid, formic acid or trifluoroacetic acid (TFA). In some aspects, the acid is a weak acid. As used herein, the term “weak acid” refers to an acid with a low concentration and/or a lower acid dissociation constant (Ka). Weak acids only partially dissociate, as compared to strong acids which fully dissociate. This corresponds to the dissociation constant (Ka) of the acid, which represents the equilibrium constant for the dissociation reaction of the acid in aqueous solution when the acid donates a proton (H+) to water to form its conjugate base (A−) and a hydronium ion (H3O+). Weak acids are characterized by a pK in a range of about −2 to 12. Strong acids are characterized by a pK of around −2. In general, a stronger type of acid can have a lower pK than a weaker acid. Conversely, a weaker type of acid can have a higher pK than a stronger type of acid. Exemplary weak acids include, but are not limited to, citric acid (C6H8O7), acetic acid (CH3COOH), oxalic acid (HO2C2O2H), sulfurous acid (H2SO3), hydrogen sulfate ion (HSO4−), phosphoric acid (H3PO4), Pitrous acid (HNO2), hydrofluoric acid (HF), methanoic acid (HCO2H), benzoic acid (C6H5COOH), and formic acid (HCOOH). Exemplary strong acids include, but are not limited to, hydrogen chloride (HCl), nitric acid (HNO3), hydroiodic acid (HI), perchloric acid (HClO4), and chloric acid (HClO3).

In some aspects, therefore, a strong acid (e.g., HCl) can be used at a lower concentration of about 0.5N or less in the second decellularization step, and similarly a weak acid may be used at a higher concentration to obtain the same results. In some instances, the second decellularization solution may comprise 0.5 N hydrochloric acid.

In an embodiment, the concentration range of the acid may be between about 0.01 M to about 5 M. Thus, in an embodiment, the acid can be used at a concentration of about 0.01 M to about 0.05 M, about 0.05 M to about 0.1 M, about 0.1 M to about 0.5 M, about 0.5 M to about 1 M, about 1 M to about 1.5 M, about 1.5 M to about 2 M, 2 M to about 2.5 M, about 2.5 M to about 3 M, about 3 M to about 3.5 M, about 3.5 M to about 4 M, 4 M to about 4.5 M, or about 4.5 M to about 5 M.

In some aspects, the second decellularization solution comprises a base. In some aspects, the base is a weak base, or a low concentration of a strong base. As used herein, the term “weak base” refers to a base with a lower base dissociation constant (Kb). Weak bases only partially accept protons in aqueous solution, as compared to strong bases, which react almost completely. This corresponds to the base dissociation constant (Kb), which represents the equilibrium constant for the reaction of the base (B) with water to form its conjugate acid (BH⁺) and a hydroxide ion (OH⁻). Weak bases are typically characterized by a pkb value in the range of about 2 to 12. Strong bases generally have pKb values less than about 2. In general, a stronger base has a lower pKb than a weaker base, whereas a weaker base has a higher pKb than a stronger base. Exemplary weak bases include, but are not limited to, ammonia (NH₃), methylamine (CH₃NH₂), aniline (C₆H₅NH₂), pyridine (C₅H₅N), trimethylamine (N(CH₃)₃), and bicarbonate ion (HCO₃⁻). Exemplary strong bases include, but are not limited to, sodium hydroxide (NaOH), potassium hydroxide (KOH), lithium hydroxide (LiOH), barium hydroxide (Ba(OH)₂), and calcium hydroxide (Ca(OH)₂). The base can be used as a concentration of about 0.01 M to about 5 M. In an aspect, the base concentration is about 0.1 M to about 1 M. Thus, in an embodiment, the base can be used at a concentration of about 0.01 M to about 0.05 M, about 0.05 M to about 0.1 M, about 0.1 M to about 0.5 M, or about 0.5 M to about 1 M.

In the second decellularization process, the cleaned particulate tissue is placed in an acid or base solution, and the tissue is allowed to swell. Blending, homogenization, or other mixing techniques can be used to break apart the tissue fragments and aid in solubilization of the collagen, gelatin, growth factors, and ECM. Once a homogenous solution is obtained, the second step decellularization solution can be used as is or filtered. The unfiltered second step decellularization solution can also contain residual, insoluble birth tissue particulate. The second step decellularization solution is then neutralized with an appropriate neutralizing acid or neutralizing base solution. Neutralization can be done using any one or more of the acids or bases provided above or known in the art. The desired pH after neutralization may depend on the application but typically ranges from about pH 4 to about pH 8.

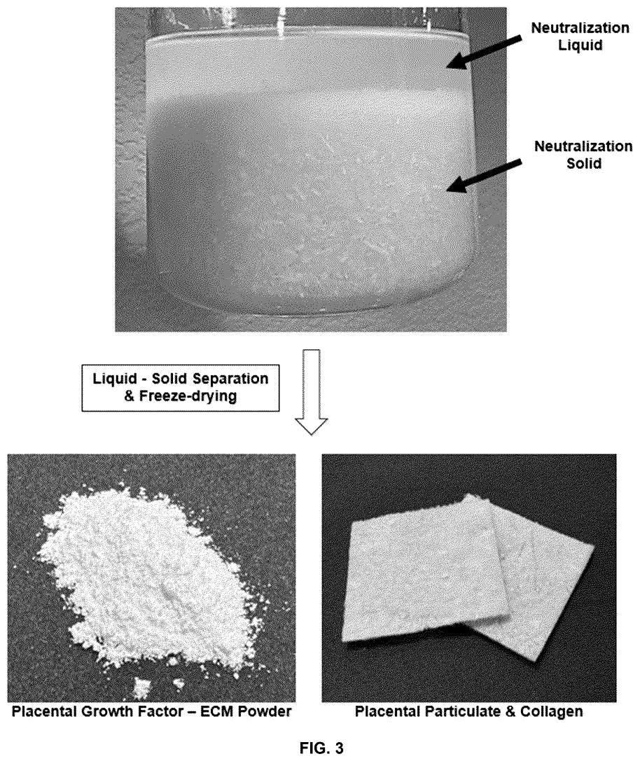

Following neutralization, the mixture separates into a neutralization solid (consisting of residual particulate placenta and precipitated materials (e.g., collagen)) and a neutralization liquid (e.g., water soluble growth factors and ECM). The neutralization liquid is seen as the opaque solution above the neutralization solid in FIG. 2 and FIG. 3. In some aspects, at this stage of the process, two drying options can be used. These drying options can include air drying, freeze drying, dehydration, or other methods known in the art. Some drying options are indicated in the process flowchart in FIG. 1. In one drying option, the neutralization solid and liquid can be freeze-dried together into a placental collagen-growth factor-ECM sheet. This sheet can be further processed after drying using a mill or blender to create a placental collagen-growth factor-ECM powder. In another drying option (FIG. 3), the neutralization solid can be separated from the neutralization liquid by decanting techniques, centrifugation, sieving, or other liquid-solid separation methods. The neutralization solid can be washed with water and the water rinses can be combined with the separate neutralization liquid. Once separated, the neutralization solid and the neutralization liquid can be freeze-dried independently This creates a placental collagen sheet (composed of residual placental particulate and precipitated collagen) and a placenta growth factor-ECM foam. Both the sheet and foam can be milled or blended after drying to create powders. In another embodiment, the isolated neutralization solid can be partially or fully converted into gelatin or hydrolyzed collagen through a third acid or base decellularization process. This process can be conducted according to methods known in the art, including extended acid or base treatments, acid or base treatment with higher concentrations, and/or heat treatments. The gelatin fabrication process can be conducted on all the collagen isolated from the birth tissue or a portion of the collagen to create both birth-tissue derived collagen and gelatin. This results in a placental gelatin sheet which can be further milled into a placental gelatin powder.

In another embodiment, the neutralization liquid can be subject to additional processing. This includes purification, separation, and/or concentration by using protein purification and desalting techniques (e.g., molecular weight cutoff filtration). The materials can then be dried using methods such as freeze-drying.

In another embodiment, the neutralization solid and/or liquid can be cast into a mold prior to freeze drying to create a variety of shapes and sizes. In another embodiment, the freeze-dried neutralization liquid can be reconstituted in a liquid with a certain weight to volume ratio to create suspensions with specific concentrations (for example, about 1%, 2%, 3%, 4%, 5%, 6%, 7%, 8%, 9%, 10% w/v, or any range derivable therein). These suspensions can be used to coat other wound healing products including amniotic membrane and collagen wound dressings. Following coating with the suspension, the resulting product would be subjected to an additional drying step. Additionally, in some aspects, the liquid may be added to products like wound bandages, dressings, or soft tissue repair meshes, to enhance their healing capabilities.

In an embodiment, the amniotic membrane that is separated from the placental tissue can be processed independently using known amniotic tissue processing techniques. The resulting amniotic tissue, in a wet or dry form, can be combined with placental connective tissue materials to create an allograft comprising both amniotic and placental tissue.

### III. Products

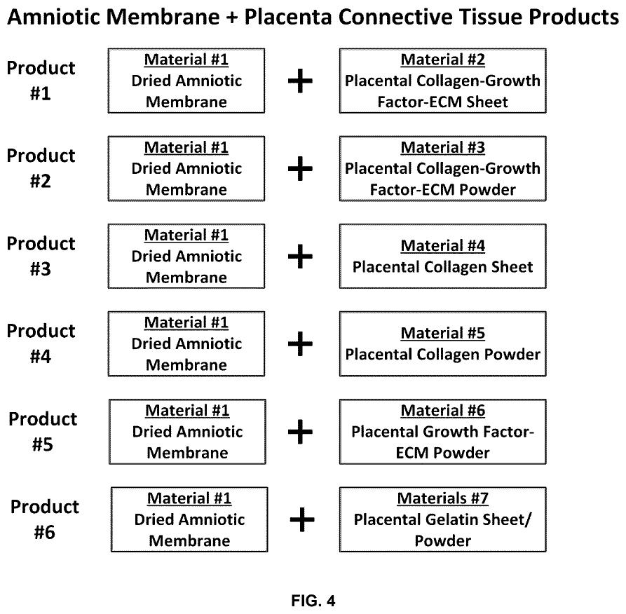

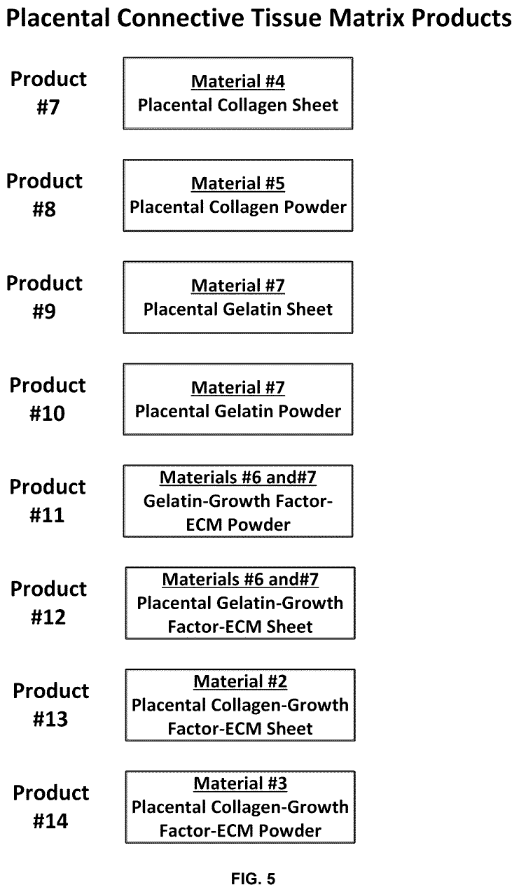

Using the techniques disclosed herein, various types of biologically active birth tissue products can be created as described in FIG. 4 and FIG. 5. In some aspects, the product is an allograft product. The term “allograft,” “allograft product,” “tissue allograft,” “allograft tissue composition” or “birth tissue allograft product” are used interchangeably and encompass a human tissue in a processed or unprocessed form that is harvested from one person and implanted or used to treat another person. In an aspect, the product may comprise other materials that are not derived from the same species, for example bovine collagen. In some embodiments, these birth tissue allografts can be used in combination with one another or other soft tissue healing devices (e.g., those based on bovine or porcine collagen or gelatin) to create a variety of products.

In some embodiments, the birth tissue allograft compositions or products comprise one or more of extracellular matrix (ECM), collagen, growth factors, or gelatin, wherein the ECM, collagen, growth factors, and/or growth factors are obtained from human birth tissue. In some embodiments, the one or more of extracellular matrix (ECM), collagen, growth factors, or gelatin are obtained from human birth tissues using a decellularization process described herein. In some embodiments, the decellularization process is the two-step process described herein. In some embodiments, the two-step process further comprises a neutralizing step as also described herein. Other steps described herein may also be included in the decellularization process, in some embodiments.

In some embodiments, the disclosure involves allograft tissue compositions or products that comprise a processed birth tissue. The birth tissue may be processed using a method disclosed herein. For example, in some embodiments, the birth tissue is processed using the two-step decellularization process disclosed herein. The decellularization process may also include a neutralizing step as disclosed herein. In some embodiments, other steps described herein may also be included in the decellularization process. In some embodiments, the processed birth tissue is obtained from one or more products (e.g., tissues, solutions, etc.) of the decellularization process. By way of a non-limiting example, in some instances the processed birth tissue is obtained from the neutralization solid, the neutralization liquid, or both the neutralization solid and the neutralization liquid. Once obtained, the processed birth tissue may be incorporated into an allograft tissue composition or product.

In some aspects, the processed birth tissue may comprise one or more of extracellular matrix (ECM), collagen, growth factors, or gelatin, wherein the ECM, collagen, growth factors, and/or growth factors.

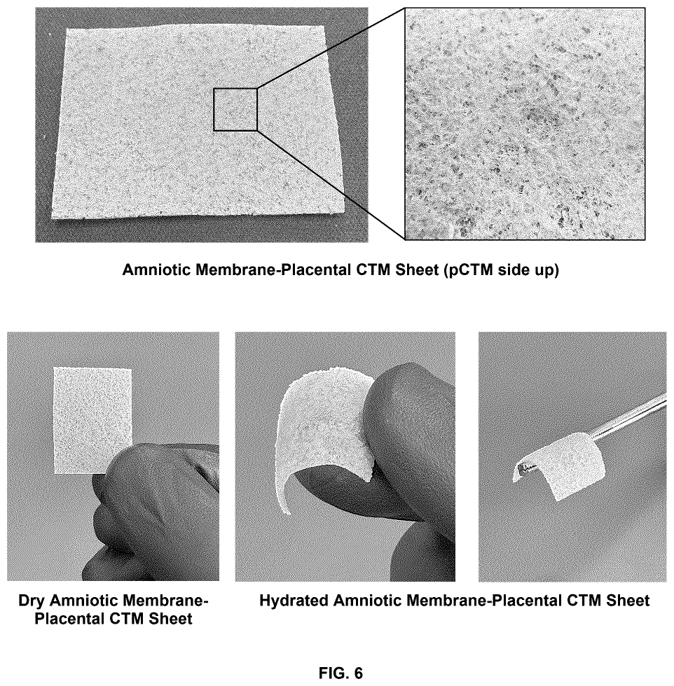

As used herein, the term “product” may refer to tissue forms that are ground, pulverized, or morselized, or provided in a sheet, powder, or gel form. In some embodiments, the birth tissue product is a sheet. In some embodiments, the birth tissue product is a powder. In some embodiments, the birth tissue allograft product is a membrane coated with a sheet or powder. In some embodiments, one or more of these products may be hydrated. In some embodiments, the hydrated birth tissue allograft product can form a gel. In some embodiments, the birth tissue allograft product is derived from placental amniotic membrane, umbilical cord, umbilical cord amniotic membrane, chorion, amnion-chorion, placenta, or any combination thereof. In the example shown in FIG. 4, amnion-placental CTM product combinations are shown. These combinations creates a new type of birth tissue allograft that provides the advantages of both amnion and placenta materials. These products include an enhanced amniotic membrane with the addition of various placental growth factor-ECM materials created from the two-step decellularization process. The benefit of these combinations is that growth factor levels can be significantly increased higher than the starting amniotic membrane to create an enhanced amniotic-placental tissue graft. Another combination includes an amniotic membrane with a placental collagen layer. In a third example, a combined collagen, gelatin, growth factor, and ECM materials can be added to an amniotic membrane. An additional benefit is that the addition of placental CTM materials to a thin 20-50 μm amniotic membrane significantly improves the product's clinical handling. This is shown in FIG. 6.

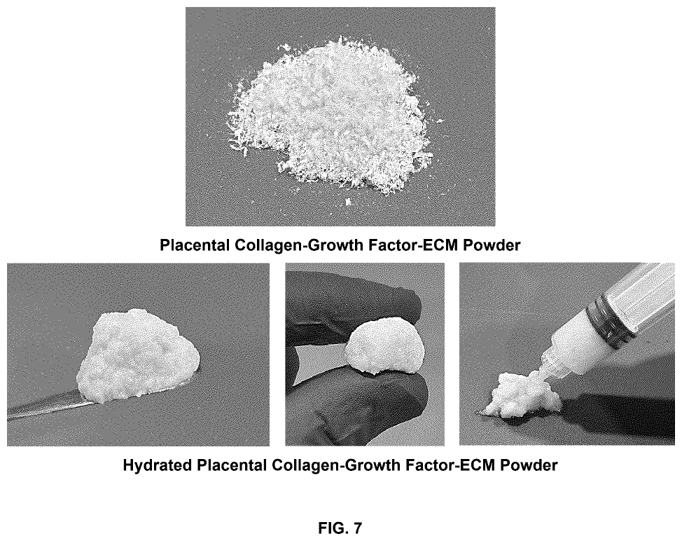

In another embodiment, the placental connective tissue matrix materials can function as standalone allograft products with no amniotic membrane. Some exemplary embodiments are also shown in FIG. 5. In this embodiment, the placenta would be processed separately and independently from standard amniotic membrane. In one example, a placental collagen-growth-ECM sheet/powder can be created (FIG. 7). Other examples include placental collagen sheet/powder or placental growth factor-ECM powders. Additional examples include additional processing of placental collagen into placental gelatin sheets and powders. One advantage of placental materials derived from a 2-step decellularization process is that growth factor levels are maintained due to an initial gentle decellularization step followed by a second acid/base step that fully captures all soluble materials. Additionally, the second acid/base decellularization step can solubilize a portion of the growth factors and ECM from the placental tissue particulate and improve tissue solubility. When freeze dried together with placental collagen and residual placental particulate, the growth factor ECM material coats the placental collagen and residual placental particulate. When used to treat a wound or soft tissue injury, this coating makes it easier for the growth factor-ECM compounds to solubilize in body fluid. As such, the availability of the tissue is increased.

Another advantage of these types of allograft products is that they may be based on human birth tissue, rather than bovine or porcine sources. This avoids issues arising from patients with allergies to bovine or porcine products, or who do not want animal products used in their treatment. In addition, the use of collagen from the placenta results in an improved collagen combination consisting of multiple collagen types (including type I, type III, and type IV) that are beneficial to soft tissue healing and are not found in standard animal-derived collagen wound products. Further, birth tissue allograft products containing growth factors and ECM provides increased biological activity compared to collagen only products, which lack these compounds. An additional advantage is that by processing both the amniotic membrane and the placenta, the amount of beneficial allograft products that can be derived from a single donor is increased.

The human birth tissue materials giving rise to the human birth tissue allograft products of the present disclosure may also provide improved handling characteristics useful for placement and implantation. In one embodiment, the human birth tissue allograft product, for example a sheet may be flexible and readily conforms to multiplanar shapes (e.g., can conform to the geometry of the wound, defect or application site). In certain embodiments, the human birth tissue sheet may be of various physical sizes, thicknesses, and shapes. This provide an improvement over standard amniotic membrane which are limited by the starting tissue thickness and size. According to such an embodiment, the human birth tissue sheet is preferably of sufficient size and shape to be applied onto or around a wound or defect. The human birth tissue sheet thickness may vary depending on the composition(s), the number of layers, and the intended use. In certain embodiments, the human birth tissue sheet may readily conform to the geometry of an application site. The size of the human birth tissue sheet may be adjusted to a desired size and shape at the time of implantation by a medical professional using any method known in the art (e.g., trimming and suturing).

In another embodiment, the birth tissue product is in a powder form. According to such an embodiment, the birth tissue powder can be added directly to a tissue repair site or can be hydrated prior to application to form a paste or gel. Pre-hydrated paste and gel forms are advantageous in that the material can adhere to a wound or soft tissue injury site allowing for accurate placement. Additionally, flowable paste and gel forms can be delivered using a syringe with an attached cannula or needle. This allows for delivery in difficult to reach sites.

In another embodiment, the birth tissue product is a combination of amniotic membrane and placental CTM materials. Due to the small thickness of amniotic membrane (20-50 μm), the tissue is difficult to handle and easily folds on itself when hydrated. While double, triple, or quadruple layer membranes can be created this significantly lowers the yield from the starting amniotic membrane and only offer slight improvements to handling. Addition of placental CTM materials to an amniotic membrane can significantly improve clinical handling during placement, as shown in FIG. 6. The hydrated membrane is flexible, can be picked with fingers, and can be easily placed at the treatment site.

In another embodiment, the birth tissue product is a placental collagen—growth factor—ECM powder. This is shown in FIG. 7. In a particulate form, the placental CTM material can be hydrated to form a moldable paste that can be handled with fingers or extruded from a syringe. This improved handling form is not possible with amniotic membrane, due to its tough membrane consistency.

Apart from the greater availability, enhanced solubility, and/or concentration of beneficial materials, the products disclosed herein may be tailored to have a monophasic or biphasic release profile depending on the degree of exposure of the birth tissue to the second decellularization step. For instance, for wound healing products, it may be better to have the product comprise an increased portion of readily soluble materials (for example, by incorporating higher amounts of the neutralization liquid that is obtained after the second decellularization step), as changes of wound dressings are frequent. Similarly, for allograft products implanted at a surgery site, it may be better to have the product comprise an increased portion of the insoluble materials (for example, by incorporating higher amounts of the neutralization solid that is obtained after the second decellularization step). This product would provide an initial release of beneficial compounds from the incorporated soluble materials followed by a slow release of materials embedded in the residual tissue particulate. This biphasic release profile and the flexibility of product design are added advantages of the disclosed method.

In some aspects, the biphasic release profile or the monophasic release profile may comprise a fast release phase that lasts for less than about 1, 2, 3, 4, 5, 6, 7 or 24 hours (or any range derivable therein). In some aspects, the biphasic release profile may comprise a slow phase lasts for greater than about 2-7 days, 1-2 weeks, 2-4 weeks (about 1 month), 1-2 months, 2-3 months, 3-4 months, or more.

Some exemplary allograft products are further described below:

In some aspects, the allograft product is a sheet composed of placental tissue processed using a two-step decellularization method that preserves the original levels of endogenous collagen, growth factors, and extracellular matrix (ECM) compounds. The sheet form allows for direct application to wounds, burns, or surgical sites, providing a scaffold that supports new tissue formation and delivers a concentrated dose of biologically active molecules. The sheet can be tailored in thickness and size to conform to various wound geometries and can be further processed into flexible, multi-planar configurations for diverse clinical applications. Depending on the application, the sheet may be formulated with a higher concentration of instant release, soluble compounds.

In some aspects, the allograft product is in a powder or particulate form that is created by blending or milling freeze-dried placental CTM material that has undergone the two-step processing method. The powder contains a blend of residual birth tissue particulate, collagen, growth factors, and ECM components, offering a scaffold material with high availability and rapid solubilization when applied to moist wound environments. It is suitable for use in irregularly shaped wounds, deep tissue defects, or as an additive to other wound care products. The powder can be hydrated to form a paster, gel or suspension (FIG. 7), allowing for customizable application and release profiles.

In some aspects, the allograft product consists of a standard amniotic membrane that has been coated with placental collagen, growth factors, and ECM materials produced via the two-step decellularization process (FIG. 6). The combination leverages the barrier and scaffold properties of the amniotic membrane with the enhanced biological activity of placental compounds. In some aspects, this dual-layer laminate is designed for use in soft tissue repair, surgical grafting, and wound healing, providing both immediate and sustained release of biologically active molecules.

In some aspects, the allograft product is created by further processing placental collagen into gelatin, resulting in a gelatin sheet or powder form. Gelatin offers hemostatic properties, maintains a moist wound environment, and can be easily shaped or molded to fit complex wound topographies. The allograft product retains growth factors and ECM components, supporting accelerated healing and tissue regeneration.

In some aspects, the allograft product is a traditional collagen or gelatin wound product (which may be derived from bovine, porcine, or human sources) that is enhanced by the addition of placental collagen, growth factors, and ECM materials derived from the use of the disclosed method. The integration of birth tissue compounds increases the biological activity of the collagen or gelatin wound product, promoting faster and more effective tissue repair due to increased biological activity. In some aspects, the product can be provided in sheet, powder, or gel form, and can be tailored for rapid or sustained release of beneficial compounds depending on clinical needs.

In some aspects, the allograft product may be a foam or particulate material derived from the neutralization liquid fraction from the second decellularization step that is rich in soluble growth factors and ECM. In some aspects, it can be used as a standalone product for topical application or as an additive to other wound care products, providing a burst release of biologically active molecules to stimulate cellular healing responses.

The two-step processing method allows for the creation of allograft products with tunable release profiles. By varying the ratio of neutralization solid to neutralization liquid, or by combining different portions of the processed tissue, products can be engineered for immediate, delayed, or biphasic release of beneficial compounds. This flexibility enables the development of wound dressings, implants, or injectable formulations optimized for specific clinical scenarios, such as frequent dressing changes or long-term tissue regeneration.

In some aspects, one or more of these allograft products may be combined to form an enhanced birth tissue allograft product, wherein the product retains original levels of endogenous collagen, growth factors, and ECM compounds, exhibits increased availability of said compounds, and is configured to provide a tunable release profile of biologically active molecules-immediate, sustained, delayed, or biphasic-adapted for application to wounds, burns, surgical sites, soft tissue defects, or as an additive to other wound care materials, thereby supporting tissue regeneration, hemostasis, and accelerated healing across a range of clinical scenarios.

### IV. Applications and Methods of Treatment

In an embodiment, the current disclosure also encompasses methods of using the disclosed allograft products for treatment of a subject in need thereof. In an aspect, the current disclosure also encompasses steps for treating a subject in need thereof, using any one or more of the disclosed allograft products.

Provided herein is a method of treating a wound or defect. The wound may be a diabetic ulcer, decubitus ulcer, venous leg ulcer, arterial leg ulcer, cutaneous ulcer, or a wound arising on or around a soft tissue, nerve, organ, vascular tissue, muscle, ligament, tendon, spinal cord, oral cavity, ocular surface, or any combination thereof. According to one embodiment, the defect is a soft tissue defect. The method may comprise an initial step of cleaning the wound and/or preparing the wound site. The method may further comprise the steps of preparing or obtaining a human birth tissue allograft product according to methods provided herein and placing the allograft product on or around the wound or defect. The allograft product may be applied one or more times on the area of the wound. Thus, the allograft product may be applied at least once, twice, three times, four times, five times or more to the wounded area. The allograft product may be applied once, once daily, twice daily, weekly, bi-weekly, monthly, bi-monthly, or more during the duration of treatment. The method may further comprise monitoring the wound healing process regularly and replacing or adjusting the one or more of the disclosed allograft products as needed based on clinical progress and physician recommendations.

According to one embodiment, the method of treating a wound or defect can further comprise the step of hydrating the allograft product prior to application. The method includes adding sterile water, saline, blood, or other liquid to the human birth tissue product and mixing the liquid into the material. The hydration of the birth tissue product can allow it to form a paste, gel, putty that can be directly handled by the clinician or may be applied using syringes, cannulas, spatulas, or other surgical means.

According to another embodiment, a method of promoting regeneration of diseased or damaged tissue is provided. The method includes the steps of preparing a human birth tissue laminate according to the methods provided herein; and placing the human birth tissue laminate graft on or around the diseased or damaged tissue. According to one embodiment, the tissue is soft tissue, nerve, organ, vascular tissue, muscle, ligament, tendon, spinal cord, oral cavity, ocular surface, or a combination thereof. According to one embodiment, the method of promoting regeneration of diseased or damaged tissue can further comprise the step of hydrating the human birth tissue laminate graft prior to application.

The treatment method disclosed herein may be combined with one or more other treatments, non-limiting examples of which include use of topical or ingested antibiotics, pain medication, steroids, and other therapeutics, surgery, plasters, wound dressings, surgical matrices, etc.

### V. Kits

According to another embodiment, a kit for use by a medical professional is provided. The kit includes one or more packaged and sterilized human birth tissue products as provided herein. The kit may also include delivery instruments such as spatulas, syringes, cannulas, needles, or the like. The kit may further include at least one set of instructions.

### Example

The following non-limiting additional examples of manufacture are provided for illustrative purposes to facilitate a more complete understanding of the representative example instances. These examples should not be construed to limit any of the example instances described in the present specification. In the following descriptions, a birth tissue process and various tissue repair compositions using the same are described for such illustrative purposes.

### Example 1:2-Step Placental Processing Method

In an embodiment, the current disclosure provides a method of processing birth tissue to maintain original levels of endogenous growth factors and ECM, and increase the bioavailability of these compounds. The method comprising rinsing the birth tissue to remove surface blood, debris, and cellular components, exposing the birth tissue to an initial gentle decellularization solution, exposing the birth tissue to a second acid or base decellularization solution, neutralizing the second decellularization solution, drying the solid and liquid portions to form birth tissue sheets or powders. A method for creating beneficial materials from placental tissues was developed and tested as described.

An overview of the steps for processing placenta are provided in FIGS. 1 and 2. As a first step, the amniotic membrane was removed, and the placenta was frozen and subsequently diced with a stainless-steel serrated blade into ˜¼″ cubes. The placental cubes were rinsed in water until a clear rinse solution was observed. The rinsed tissue was placed in a multi-blade blender and chopped using the pulse function. The particulate tissue was placed on a 212 μm sieve and further rinsed with water until a clear rinse was observed. Excessive water was removed, and the wet tissue particulate was added to a 2000 ml beaker. An initial decellularization was conducted by adding 800 ml of 4% Triton X-100 detergent solution to the particulate tissue. The mixture was stirred with an overhead stirrer at a 500 RPM stir speed for 2 hours. Following stirring, the Triton-X solution was removed, and fresh Triton X-100 solution (800 ml) was added. The mixture was allowed to soak for 16-24 hours in a refrigerator at ˜4° C. without stirring. Following soaking, the tissue had a light tan appearance. The tissue was poured onto a 212 μm sieve and rinsed with water to remove the detergent. This was conducted until detergent foaming was no longer observed. Following rinsing, excess water was squeezed out of the tissue. A second decellularization step was conducted by suspending the particulate tissue in 0.1 N HCl using a ratio of 1 g of wet tissue to 3 ml of 0.01 N HCl. Following the addition of 0.1 N HCl, the tissue was initially homogenized and then stirred in the acid for 2 hours. At 30 minutes, 1 hour, and 2 hours of mixing, the tissue was subjected to additional homogenization steps. After 2 hours of stirring, the tissue-acid mixture consisted of an opaque solution and residual placenta particulate. The mixture was then neutralized with addition of 5 M NaOH until a pH 7-8 was obtained. As the NaOH was added, a precipitate formed in the solution (also referred herein as the neutralization solid). This combined with the residual placental particulate. The remaining solution (also referred herein as the neutralization liquid) had an opaque appearance, indicating the presence of soluble growth factors and ECM. The neutralization solid was separated from the neutralization liquid, and the solution was saved. The neutralization solid was rinsed with water and the water rinse was added to the separate neutralization liquid. The wet neutralization solid was pressed into a sheet form using a mold. The neutralization liquid was placed in a separate freeze-drying tray. Both the neutralization solid material and the neutralization liquid were frozen and separately freeze-dried to remove residual water. This resulted in a placental tissue/collagen sheet and a growth factor/ECM particulate (flowing blending). This is shown in the bottom images of FIG. 3.

### Example 2: Preparation of a Placental Collagen-Growth Factor-ECM Sheet

A second example of the technology disclosed herein can include methods for creating a collagen-growth factor-ECM sheet and powder, as exemplified in the example below.

The process of Example 1 was carried out to the neutralization step. The neutralization solid and neutralization liquid were briefly mixed to create a homogenous suspension. The wet collagen-growth factor-ECM dispersion was placed in a sheet mold with a ˜3 mm height. The mixture was frozen, and freeze dried to create a sheet. The sheet was cut with scissors into individual wound dressings with a ˜2″×2″ size.

### Example 3: Preparation of a Placental Collagen-Growth Factor-ECM Powder

A third example of the technology disclosed herein can include methods for creating a collagen-growth factor-ECM wound powder, as exemplified below.

For this, following the process of Example 2, the placental collagen-growth factor-ECM sheet was blended with a multi-blade blender to produce a powder. The resulting material is shown in FIG. 7.

### Example 4: Preparation of an Amniotic Membrane Coated with Placental Collagen-Growth Factor-ECM

A fourth example of the technology disclosed herein can include methods for creating a birth tissue laminate consisting of an amniotic membrane coated with placental collagen-growth factor-ECM sheet, as exemplified in the example below.

A collagen-growth factor-ECM sheet from Example 2 was cut to a size slightly larger than a dried amniotic membrane. The collagen sheet was fully hydrated with water. A dry amniotic membrane was placed on top of the hydrated collagen sheet. Contact of the dry amniotic membrane with the hydrated collagen sheet caused the amniotic membrane to hydrate. The layers were pressed together and allowed to air-dry. Following air-drying, the collagen-growth factor-ECM sheet adhered to the amniotic membrane. This resulted in an amniotic membrane on one side and a collagen-growth factor-ECM sheet on the other. This is shown in FIG. 6.

It is to be understood that the specific order or hierarchy of steps in the method(s) of this disclosure are instances of example approaches and can be rearranged while remaining within the disclosed subject matter. For instance, any of the operations discussed throughout this disclosure may be omitted, repeated, performed in parallel, performed in a different order, and/or combined with any other of the operations of this disclosure.

While the present disclosure has been described with reference to various implementations, it will be understood that these implementations are illustrative and that the scope of the present disclosure is not limited to them. Many variations, modifications, additions, and improvements are possible. More generally, implementations in accordance with the present disclosure have been described in the context of particular implementations. Functionality may be separated or combined differently in various implementations of the disclosure or described with different terminology. Any of the components or steps disclosed herein can be duplicated, omitted, and/or combined with any other components or steps disclosed herein. These and other variations, modifications, additions, and improvements may fall within the scope of the disclosure as defined in the claims that follow

1. A method of processing a birth tissue, the method comprising:
a) obtaining or having obtained the birth tissue;
b) subjecting the birth tissue to a two-step decellularization process, comprising:
i. a first decellularization step comprising exposing the birth tissue to a first decellularization solution to remove blood-based cells and/or blood materials to obtain a clean tissue; and
ii. a second decellularization step comprising exposing the clean tissue to a second decellularization solution, comprising an acid or a base, to obtain a second step decellularization solution;
c) neutralizing the second step decellularization solution to a pH of 4-8 to obtain a neutralization solid and a neutralization liquid.

2. The method of claim 1, wherein the method further comprises an initial size reduction step for the birth tissue prior to subjecting the birth tissue to the two-step decellularization process to obtain a particulate form of the birth tissue.

3. (canceled)

4. The method of claim 2, further comprising cleaning the birth tissue or the particulate form of the birth tissue with a cleaning agent.

5. (canceled)

6. The method of claim 1, wherein the birth tissue is harvested from umbilical cord, amniotic sac, or placenta, or any combination thereof, and wherein the birth tissue comprises placental globe, umbilical cord, umbilical cord vessels, umbilical cord blood, chorionic membrane, amniotic membrane, Wharton's jelly, amniotic fluid, or extracellular material, or any combination thereof.

7. (canceled)

8. The method of claim 1, wherein the birth tissue is derived from a human.

9. The method of claim 1, wherein the first decellularization solution comprises water, salt solutions, ionic detergents, enzymes, or alcohol solutions, or any combination thereof.

10. The method of claim 9, wherein the ionic detergent is Triton X-100.

11. The method of claim 9, wherein the enzymes comprise trypsin, DNAase, collagenase, dispase, or lipase, or any combination thereof.

12. The method of claim 1, wherein second decellularization solution comprises an acid solution comprising hydrochloric acid, acetic acid, citric acid, lactic acid, formic acid, or trifluoroacetic acid (TFA) or a base solution comprising sodium hydroxide, ammonium hydroxide, potassium hydroxide, or calcium hydroxide, wherein the acid solution or the base solution has an acid concentration or base concentration respectively ranging from 0.01 M to 5 M.

13-14. (canceled)

15. The method of claim 1, wherein the neutralizing step (c) comprises:
(i) a neutralizing acid or neutralizing base solution that comprises: a base solution comprising sodium hydroxide, potassium hydroxide, calcium hydroxide, sodium carbonate, ammonium hydroxide, or any combination thereof, or an acid solution comprising hydrochloric acid, acetic acid, citric acid, lactic acid, formic acid, trifluoroacetic acid (TFA), or any combination thereof, wherein the neutralizing acid or neutralizing base solution has an acid concentration or base concentration respectively ranging from 0.01 M to 10 M, or
(ii) a neutralization process utilizing dialysis, diafiltration or other means.

16-20. (canceled)

21. The method of claim 1, further comprising: separating the neutralization solid from the neutralization liquid and drying the neutralization solid and/or the neutralization liquid.

22-25. (canceled)

26. The method of claim 21, wherein the drying is a freeze-drying, air drying, and/or dehydration process.

27. (canceled)

28. The method of claim 21, wherein the dried neutralization solid and/or the neutralization liquid is incorporated into an allograft tissue composition, wherein the allograft tissue composition has increased solubility for one or more of collagen, gelatin, growth factors, or extracellular matrix compounds.

29-37. (canceled)

38. An allograft tissue composition comprising a processed birth tissue, wherein the birth tissue is processed using a method comprising:
a) obtaining or having obtained the birth tissue;
b) subjecting the birth tissue to a two-step decellularization process, comprising:
iii. a first decellularization step comprising exposing the birth tissue to a first decellularization solution to remove blood-based cells and/or blood materials to obtain a clean tissue; and
iv. a second decellularization step comprising exposing the clean tissue to a second decellularization solution, comprising an acid or a base, to obtain a second step decellularization solution;
c) neutralizing the second step decellularization solution to a pH of 4-8 to obtain a neutralization solid and a neutralization liquid,
wherein the processed birth tissue is obtained from the neutralization solid, the neutralization liquid, or both the neutralization solid and the neutralization liquid.

39. The allograft tissue composition of claim 38, wherein the neutralization solid comprises a collagen precipitate and/or residual birth tissue particulate.

40. (canceled)

41. The allograft tissue composition of claim 38, wherein the method further comprises separating the neutralization solid from the neutralization liquid.

42. (canceled)

43. The allograft tissue composition of claim 41, further comprising drying the neutralization solid and/or the neutralization liquid and incorporating the dried neutralization solid and/or the neutralization liquid into the allograft tissue composition.

44. (canceled)

45. The allograft tissue composition of claim 43, wherein the drying is a freeze-drying, air-drying, and/or dehydration process.

46. The allograft tissue composition of claim 38, wherein the allograft tissue composition is a sheet, a powder, a gel, a paste, or any combination thereof.

47. The allograft tissue composition of claim 38, wherein the allograft tissue composition has increased solubility for one or more of collagen, gelatin, growth factors, and extracellular matrix compounds.

48. The allograft tissue composition of claim 38, wherein the allograft tissue composition releases, within less than about 1 hour to less than about 8 hours, one or more of growth factors and/or extracellular matrix compounds.

49. The allograft tissue composition of claim 38, wherein the allograft tissue composition has a biphasic release rate, with a first fast phase and subsequent slow phase for growth factors and/or extracellular matrix compounds; wherein the first fast phase lasts for less than about 1 hour to less than about 8 hours and the slow phase lasts for greater than about 2 days to greater than 6 months.

50-55. (canceled)

56. A birth tissue allograft product, comprising one or more of extracellular matrix (ECM), collagen, growth factors, or gelatin, wherein the ECM, collagen, growth factors, and/or growth factors are obtained from human birth tissue using a two-step decellularization process, wherein the two-step decellularization comprises:
i. exposing the human birth tissue to a first decellularization solution to remove blood-based cells and/or blood materials to obtain a clean tissue; and
ii. exposing the clean tissue to a second decellularization solution, comprising an acid or a base, to obtain a second step decellularization solution.

57-58. (canceled)

59. The birth tissue allograft product of claim 56, wherein the birth tissue comprises placental globe, umbilical cord, umbilical cord vessels, umbilical cord blood, chorionic membrane, amniotic membrane, Wharton's jelly, amniotic fluid, or extracellular material, or any combination thereof.

60. (canceled)

61. The birth tissue allograft product of claim 56, wherein the allograft tissue product releases, within less than about 1 hour to less than about 8 hours, one or more of growth factors and/or extracellular matrix compounds.

62. The birth tissue allograft of claim 56, wherein the allograft tissue product has a biphasic release rate, with a first fast phase and subsequent slow phase for growth factors and/or extracellular matrix compounds; wherein the first fast phase lasts for less than about 1 hour to less than about 8 hours and the slow phase lasts for greater than about 2 days to greater than 6 months.

63-64. (canceled)

65. The birth tissue allograft product of claim 56, wherein the allograft tissue product is a sheet, a powder, a gel, or a paste, or any combination thereof.

66. The birth allograft tissue product of claim 56, wherein the allograft tissue product further comprises an amniotic membrane.

67. The birth allograft tissue product of claim 65, wherein the allograft tissue product is added to a collagen or gelatin wound product.

68. The birth tissue allograft product of claim 56, wherein the tissue allograft product comprises at least one of the following allograft tissue compositions: (a) a placental collagen-growth factor-extracellular matrix (ECM) sheet; (b) a placental collagen-growth factor-ECM powder; (c) an amniotic membrane coated with placental collagen, growth factors, and/or ECM; (d) a placental gelatin sheet; (e) a placental gelatin sheet powder; (f) a collagen or gelatin wound product enhanced with placental collagen, growth factors, and ECM; (g) a growth factor-ECM foam or particulate.
